(19) United States
(12) Patent Application Publication
(10) Pub. No.: US 2012/0259425 A1
(43) Pub. Date: Oct. 11, 2012

(54) Precision Shaped Compressed Demineralized Cancellous Bone Product and Method to Make Same

(75) Inventor: Timothy R. BRAHM, Germantown, TN (US)

(73) Assignee: Dynamic Surgical Solutions, Germantown, TN (US)

(21) Appl. No.: 13/358,531

(22) Filed: Jan. 26, 2012

Related U.S. Application Data
(60) Provisional application No. 61/436,476, filed on Jan. 26, 2011.

(51) Int. Cl. A61F 2/28; B29C 35/16; B26D 7/10

(52) U.S. Cl. 623/23.51; 83/15; 264/28

(57) ABSTRACT

The precision formed compressed demineralized cancellous bone matrix product is made by the process of: (a) providing a demineralized cancellous bone matrix in a compression mold for the tissue implant; (b) freeze-drying the demineralized cancellous bone matrix in the mold to form freeze-dried compressed demineralized cancellous bone matrix; (c) compressing the matrix contained within the compression mold for a time and under conditions sufficient to form a tissue implant part there from; and (d) removing the precision formed compressed demineralized cancellous bone matrix product. In an alternative embodiment, the precision formed compressed demineralized cancellous bone matrix product is made by: (a) providing a compressed demineralized cancellous bone matrix; (b) freeze-drying the compressed demineralized cancellous bone matrix to form freeze-dried compressed demineralized cancellous bone matrix; and (c) cutting said compressed demineralized cancellous bone matrix to form tissue implant or tissue implant part. The precision formed compressed demineralized bone matrix product can promote healing.

### FIELD OF THE INVENTION

This invention relates to a precision shaped demineralized cancellous bone matrix product, methods to make this product and methods to use this product.

### BACKGROUND OF THE INVENTION

The use of bone products to promote healing after a facture, bone loss, infection or other pathological conditions is well known to those skilled in the relevant art. More than 500,000 bone graft procedures are performed in the United States each year and approximately 2.2 million worldwide. The estimated cost of these procedures approaches $2.5 billion per year. In most of these procedures, either autograft or allograft tissue is used. Currently for autograft tissue bone grafts, the tissue usually from the iliac crest, but also from the distal femur or the proximal tibia. The graft is then placed at the injury or operative site. This tissue is ideal as a bone graft because it possesses all of the characteristics necessary for new bone growth. The allograph tissue from demineralized bone matrix products is generally mixed with putty and gel void fillers.

In humans, the spine has seven cervical, twelve thoracic and five lumbar segments. The bony vertebral bodies of the spine are separated by intervertebral discs. The typical vertebra has a thick anterior bone mass called the vertebral body, with a vertebral arch that arises from the posterior surface of the vertebral body. The spinal disc and/or vertebral bodies may be displaced or damaged due to trauma, disease, degenerative defects, or wear over an extended period of time. One result of this displacement to a spinal disc or vertebral body may be chronic back pain. In many cases, to alleviate back pain, the disc is removed along with all or part of at least one neighboring vertebrae and is replaced by an implant that promotes fusion of the remaining bones. In this procedure, a fusion element such as a spacer, implant or cage is used to fill the space left by the removed disc and bony anatomy. A spacer includes an area for the placement of a bone graft to enhance the fusion between the adjacent vertebrae.

Osteoinductive interbody support spacers for cervical and lumbar applications are known in the art. These osteoinductive interbody support spacers are used with cervical implants. Cervical implants are devices surgeons use to decompress and stabilize the spine. These devices are implanted either from the anterior of the spine, or from the posterior. In this procedure cages and spacers are placed between two vertebrae. Their purpose is to: maintain space between vertebrae and also to preserve spinal alignment and to promote spinal fusion.

Cages come in different shapes and sizes; some are cylinder-shaped and others box-shaped. Cages are fit into the spine between vertebrae. Usually, cages are made from bone, metal, plastic, or carbon fiber. Bone chips (autograft, allograft, other bone graft substitutes, or other bone growth stimulating substances (e.g., demineralized bone matrix) may be packed into the cage. After surgery, the cage facilities the fusion between the vertebrae, and hence, increases spinal stability.

Lumbar implants are devices surgeons use to decompress and stabilize the spine. Implants used in lumbar fusion surgery can be divided into two groups: Those placed within the interbody space and those placed onto the spine to provide stabilization. The purpose of lumbar stabilization implants is to restore and maintain normal alignment of the lumbar spine and to keep the spine stable during the fusion process.

Currently, there are literally dozens of different shapes of osteoinductive material to fill the fusion holes for the various structural interbody fusion elements (e.g. VBRs, allograft) on the market. There exist numerous heights, typically eight-ten for the cervical spacers and more than five for lumbar spacers. A need exists in the industry for a universally shaped osteoinductive material. Furthermore, there are many surgical applications where filling bone voids with a lightly osteoinductive material that forms and fills into whatever void it fills would be advantageous.

### SUMMARY OF THE INVENTION

In one aspect, the present invention provides a compressed demineralized bone matrix product, made by the process of forming a compressed demineralized bone matrix into the desired shape and lyophilizing the shaped cancellous bone matrix into a tissue implant. A compressed demineralized bone product having a substantially clover-leafed shaped cross-section, is the preferred embodiment. This bone matrix product is wedge shaped from top to bottom. This shape is preferred because with this shape, a wide array of fusion holes can be filled.

In another aspect of the invention, a precision molded or formed demineralized cancellous bone matrix product is made by: (a) providing a demineralized cancellous bone matrix product in a compression mold for a tissue implant or tissue implant part; (b) lyophilizing the demineralized cancellous bone matrix in the mold to form a freeze-dried compressed demineralized cancellous bone matrix; (c) compressing the matrix contained within the compression mold for a time and under conditions sufficient to form a tissue implant or tissue implant part there from, and (d) removing the freeze-dried compressed demineralized cancellous bone matrix tissue implant or tissue implant part from the mold.

In another aspect of the invention, a precision formed tissue implant or tissue implant part is made by the steps of: (a) providing a compressed demineralized cancellous bone matrix; (b) freeze-drying the compressed demineralized cancellous bone matrix to form freeze-dried compressed demineralized cancellous bone matrix; and (c) cutting the compressed demineralized cancellous bone matrix to form a tissue implant or tissue implant part.

Another aspect of the invention, is a method to use compressed demineralized cancellous bone matrix product. This method includes the steps of: implanting a lyophilized compressed demineralized cancellous bone matrix product in a surgical cavity, wherein the demineralized cancellous bone matrix product expands to fill substantially all voids in the surgical cavity. This method also further involves the step of contacting the lyophilized compressed demineralized cancellous bone matrix product with an effective amount of a hydrating agent that can include a bioactive agent.

### BRIEF DESCRIPTION OF THE DRAWINGS

FIG. 1 is a perspective view of an embodiment of the implant according to the present invention.

### DETAILED DESCRIPTION OF THE INVENTION

The present invention relates to a method to make a precision molded tissue implant or tissue implant part. In this invention, demineralized cancellous bone matrix is lyophilized in a compressed state in a mold and then inserted, still freeze-dried, into a surgical space, where a hydrating agent, preferably including a bioactive agent, is applied to the compressed demineralized cancellous bone matrix (through a syringe with a needle). The compressed demineralized cancellous bone matrix expands into the void into which it was placed. The advantage is that the space will be fully filled and the expanding demineralized cancellous bone matrix will create light pressure between it and the surrounding bone. As the tissue implant expands, it touches the bone on either side of the implant which aids in the healing process.

According to this method, bone is first processed according to standard methods to obtain demineralized cancellous bone matrix. One method is disclosed in U.S. patent application Ser. No. 12/130,384, Process for Demineralization of Bone Matrix Preservation of Natural Growth Factors (hereby specifically entirely incorporated by reference).

Demineralized cancellous bone matrix is further treated to obtain a precision molded or formed shape. The mold can be made of plastic, metal and includes small pores to allow the evaporation of moisture. In the preferred embodiment, the draft angle of the mold of 0 to 5° but, as high as 15° may be used in the molds for easy release of the precision formed compressed demineralized bone matrix, after lyophillization.

In an exemplary embodiment, the precision formed compressed demineralized cancellous bone matrix can be used in spinal, reconstructive, trauma and oral surgical grafting procedures to promote bone healing and new bone growth. Allograft and xenograft tissue are lyophilized to reduce the risk of disease transmission by removing the water from the tissue to destroy all osteogenic cells. The tissues are also sterilized with gamma radiation, electron-beam radiation, or ethylene oxide.

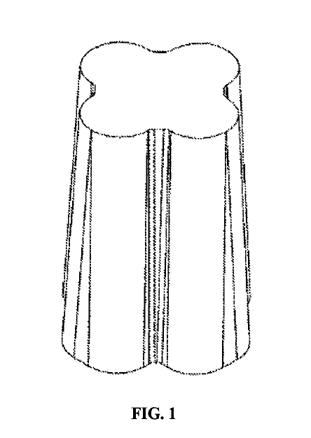

Now referring to FIG. 1, a tissue implant having a substantially clover shape in the cross-section is shown. This bone matrix product is wedge shaped from top to bottom. This shape is preferred because with this shape, a wide array of fusion holes can be filled.

In some applications, a star-shaped compressed demineralized bone matrix product can also be used. In these applications, the star shaped implant is gently pushed into the fusion hole. The points of the stars will break off during placement. The demineralized cancellous sponge is benign and is unlikely to cause any issues, if particles are left in various areas during placement.

### DEFINITIONS

Allograft is defined as tissue transplanted from one individual to another individual of the same species. However, this methodology could be also applied to cancellous bone from non-human species, i.e. equine (horse) to equine.

Bone matrix substitute encompass a variety of materials, material sources, and origins (natural vs. synthetic). Many are formed from composites of one or more types of material; however, the composite is usually built on a base material. Allograft-based bone graft substitutes involve allograft bone, used alone or in combination with other materials (eg, ALLOGRO (AlloSource, Centennial, Colo.), OPTEFORM (Exactech, Inc, Gainesville, Fla.), GRAFTON (BioHorizons, Birmingham, Ala.), ORTHOBLAST (IsoTis OrthoBiologics, Irvine, Calif.).) Polymer-based bone graft substitutes, degradable and non-degradable polymers, are used alone or in combination with other materials (eg, Cortoss [Orthovita, Inc, Malvern, Pa.], open porosity polylactic acid polymer OPLA, (Immix Osteobiologics, Inc, San Antonio, Tex.) and natural materials such as collagen. See Laurencin et al, Fracture Repair: Challenges and Opportunities, 90 J of Bone & Joint Surgery 1-2 (2008).

Bioactive agents may include, for example, antimicrobials, antibiotics, antimyobacterial, antifungals, antivirals, antineoplastic agents, antitumor agents, agents affecting the immune response, blood calcium regulators, agents useful in glucose regulation, anticoagulants, antithrombotics, antihyperlipidemic agents, cardiac drugs, thyromimetic and antithyroid drugs, adrenergics, antihypertensive agents, cholnergics, anticholinergics, antispasmodics, antiulcer agents, skeletal and smooth muscle relaxants, prostaglandins, general inhibitors of the allergic response, antihistamines, local anesthetics, analgesics, narcotic antagonists, antitussives, sedative-hypnotic agents, anticonvulsants, antipsychotics, anti-anxiety agents, antidepressant agents, anorexigenics, non-steroidal anti-inflammatory agents, steroidal anti-inflammatory agents, antioxidants, vaso-active agents, bone-active agents, osteogenic factors, antiarthritics, diagnostic agents and progenitor cells, such as stem cells. Factor-based bone graft substitutes are natural and recombinant growth factors, used alone or in combination with other materials such as transforming growth factor-beta (TGF-beta), platelet-derived growth factor (PDGF), fibroblast growth factor (FGF), and bone morphogenetic protein (BMP). Cell-based bone graft substitutes use cells to generate new tissue alone or are seeded onto a support matrix (eg, mesenchymal stem cells). Ceramic-based bone graft substitutes include calcium phosphate, calcium sulfate, and bioglass used alone or in combination (eg, OSTEOGRAPH (DENTSPLY Friadent CeraMed, Lakewood, Colo.), NORTIAN SRS (Synthes, Inc, West Chester, Pa.), PROOSTEON (Interpore Cross International, Irvine, Calif.), OSTEOSET (Wright Medical Technology, Inc, Arlington, Tenn.

Bone Matrix refers to an extracellular matrix of cancellous bone remaining after demineralization. The highly porous cancellous bone matrix provides scaffolding conducive for cell attachment and tissue regeneration.

BMPs refers to bone morphogenetic proteins that have been associated with bone and cartilage growth through the mechanism of providing signals to osteogenic cells to differentiate, proliferate and regenerate new tissue.

Cancellous bone, also called trabecular bone or spongy bone, has a higher surface area but is less dense, softer, weaker, and less stiff. It typically occurs at the ends of long bones, proximal to joints and within the interior of vertebrae. Cancellous bone may be collected from a recovered human tissue donor for allograft use.

Fusion element means spacers, implants or cage to form intervertebral fusion device.

Hydrating agent includes pharmaceutical grade water, neutral pH saline, blood, PRP.

Precision molded means a medical implant that is molded or shaped to expand to fill substantially all of the voids in a cavity.

Xenograft is defined as tissue transplanted from one species to another individual of a different species. Examples of xenograft could be equine to human, canine to feline, etc.

The following examples will illustrate the practice of the present invention in further detail. It will be readily understood by those skilled in the art that the following methods, formulations, and compositions of novel compounds of the present invention, as generally described and illustrated in the examples herein, are to be viewed as exemplary of the principles of the present invention, and not as restrictive to a particular structure or process for implementing those principles. Thus, Example 1 is not intended to limit the scope of the invention, as claimed, but is merely representative of exemplary embodiments of the invention.

### EXAMPLE 1

A method for preparing demineralized cancellous bone pieces in the present invention may include the following steps of: (1) the cancellous bone is cut into cubes approximately 10×10×10 mm or of other dimensions. It could also be cut into different shapes such as cylinders, triangular prisms, rectangles, hexagons, octagons, etc.; (2) The cancellous pieces (referred to from here as cubes, although they could be any shape) are rinsed in several solutions to remove fats, oils and other soft tissue. Other procedures may be applied such as high pressure washes and ultrasonic cleaning; (3) The cubes are rinsed; (4) The cubes undergo a series of weak acid washes and remove the minerals from the cancellous bone and leave behind the collagen matrix and much of the endogenous proteins. This leaves a spongy material that can easily be compressed with digital pressure; and (5) The cubes are rinsed in water; (6) The cubes undergo buffering steps to bring the pH up to a neutral range (around pH=7); (7) the cubes are rinsed in water; (8) The cubes are compressed into molds that take the predetermined shapes as described; (9) the cubes are lyophilized (freeze-dried) in the molds and retain the predetermined shape; (10) the cubes are removed from the molds and placed into poly-poly pouch with an allograft label and a gamma-irradiation sticker; (11) This pouch is placed in an outer poly-foil pouch; (12) The entire unit is terminally sterilized by gamma irradiation; (13) The unit is placed into the final packaging (box) with appropriate labeling.

The principles, preferred embodiments and modes of operation of the present invention have been described in the foregoing specification. However, the invention should not be construed as limited to the particular embodiments which have been described above. Instead, the embodiments described here should be regarded as illustrative rather than restrictive. Variations and changes may be made by others without departing from the scope of the present invention as defined by the following claims:

1. A precision shaped compressed demineralized cancellous bone matrix product made by the process comprising: (a) providing a demineralized cancellous bone matrix in a compression mold for a tissue implant or tissue implant part; (b) lyophilizing said demineralized cancellous bone matrix product in said mold to form a freeze-dried demineralized cancellous bone matrix product; (c) compressing the matrix contained within the compression mold for a time and under conditions sufficient to form the tissue implant or tissue implant part there from; and (d) removing a precision shaped compressed demineralized cancellous bone matrix product.

2. The product of claim 1 further comprises contacting the precision shaped compressed demineralized cancellous bone matrix product with an effective amount of a bioactive agent.

3. A precision shaped compressed demineralized cancellous bone matrix product made by the process comprising: (a) providing a compressed demineralized cancellous bone matrix (b) lyophilizing said compressed demineralized cancellous bone matrix product to form freeze-dried compressed demineralized cancellous bone matrix product; and (c) cutting said compressed demineralized cancellous bone matrix product to form a precision shaped compressed demineralized cancellous bone matrix product.

4. The product of claim 3 further comprises contacting the precision shaped compressed demineralized cancellous bone matrix product with an effective amount of a bioactive agent.

5. A method for producing a precision molded tissue implant or tissue implant part the method comprising the steps of: (a) providing a demineralized cancellous bone matrix in a compression mold for the tissue implant or tissue implant part; (b) lyophilizing said demineralized cancellous bone matrix in said mold to form a freeze-dried compressed demineralized cancellous bone matrix; (c) compressing the matrix contained within the compression mold for a time and under conditions sufficient to form a tissue implant or tissue implant part there from; and (d) removing the tissue implant or tissue implant part from the compression mold.

6. The method of claim 5, wherein the method further comprises: (e) sterilizing the tissue implant or tissue implant part using a non-irradiative process, and (f) packaging the tissue implant or tissue implant part.

7. The method of claim 5, wherein the method further comprises: (e) sterilizing the tissue implant or tissue implant part using an irradiative process, and (f) packaging the tissue implant or tissue implant part.

8. The method of claim 5 further is comprising contacting said tissue implant or tissue implant part with a bioactive agent.

9. The method of claim 5 further comprising the step of applying a hydrating agent to said compressed demineralized cancellous bone matrix prior to adding said compressed demineralized cancellous bone matrix to said mold.

10. The method of claim 5 further comprising mixing a bone matrix substitute with said compressed demineralized bone matrix.

11. A method for producing a precision formed tissue implant or tissue implant part the method comprising the steps of: (a) providing a compressed demineralized cancellous bone matrix; (b) freeze-drying said compressed demineralized cancellous bone matrix to form freeze-dried compressed demineralized cancellous bone matrix; and (c) cutting said compressed demineralized cancellous bone matrix to form tissue implant or tissue implant part.

12. The method of claim 11, wherein the method further comprises: (d) sterilizing the tissue implant or tissue implant part using a non-irradiative process, and (e) packaging the tissue implant or tissue implant part.

13. The method of claim 11, wherein the method further comprises: (d) sterilizing the tissue implant or tissue implant part using an irradiative process, and (e) packaging the tissue implant or tissue implant part.

14. The method of claim 11 further comprising contacting said tissue implant or tissue implant part with a bioactive agent.

15. The method of claim 11 further comprising the step of applying a hydrating agent to said compressed demineralized cancellous bone matrix prior to adding said compressed demineralized cancellous bone matrix to said mold.

16. The method of claim 11 further comprising mixing a bone matrix substitute with said compressed demineralized bone matrix.

17. A method to use compressed demineralized cancellous bone matrix product, the method comprising the steps of: implanting a lyophilized compressed demineralized cancellous bone matrix in a surgical cavity, wherein the demineralized cancellous bone matrix product expands to fill substantially all of voids in said surgical cavity.

18. The method of claim 17 further comprises contacting said lyophilized compressed demineralized cancellous bone matrix product with an effective amount of a hydrating agent.

19. The method of claim 17 wherein said hydrating agent includes a bioagent.
